(19) United States
(12) Patent Application Publication
(10) Pub. No.: US 2023/0037146 A1
(43) Pub. Date: Feb. 2, 2023

(54) DEVICE AND METHOD FOR EVALUATING POSTURAL CONTROL ABILITY

(71) Applicant: J-MEX INC., Hsinchu City (TW)

(72) Inventor: Deng-Huei Hwang, Hsinchu City (TW)

(21) Appl. No.: 17/867,811

(22) Filed: Jul. 19, 2022

(30) Foreign Application Priority Data
Jul. 20, 2021 (TW) 110126678

(51) Int. Cl. A61B 5/11

(52) U.S. Cl. CPC A61B 5/1116; A61B 5/1118

(57) ABSTRACT

The present invention provides a method for evaluating the postural control ability of a subject, including the steps of configuring a plurality of motion sensors on a plurality of body parts of the subject correspondingly to generate a plurality of sensing data, wherein each of the plurality of sensing data includes static sensing data and dynamic sensing data; performing a posture determination procedure according to the plurality of static sensing data to determine a specific body posture of the subject; and performing an evaluation procedure for the postural control ability. The evaluation procedure includes the steps of processing each of the plurality of dynamic sensing data to obtain a posture maintenance parameter for each of the plurality of body parts under the specific body posture, and comparing the posture maintenance parameter to a reference value of posture maintenance for each of the body parts under the specific body posture so as to generate an evaluation result of the postural control ability.

### CROSS-REFERENCE TO RELATED APPLICATION AND CLAIM OF PRIORITY

This application claims priority to Taiwan Patent Application No. 110126678 filed on Jul. 20, 2021, which are incorporated herein in its entirety by reference.

### FIELD OF THE INVENTION

The present invention is related to a device and a method for evaluating postural control ability of a subject. In particular, the present invention is related to a device and a method for automatically evaluating postural control ability of the subject.

### BACKGROUND OF THE INVENTION

Due to hemiplegia in stroke patients, the muscle strength, movement function and balance ability of the trunk and limbs are affected/paretic, which may affect the sitting and standing performances of the stroke patients. Therefore, restoring the sitting balance, the standing balance, and the sitting-to-stand limb function are not only the recovery goal for stroke patients, but also the basis for restoring the ability to walk in the future.

The purpose of static sitting rehabilitation is to reestablish a stable sitting posture and sitting balance ability for the stroke patients by practicing sitting balance training. Due to the weakness of the affected/paretic limbs and poor postural control of the stroke patients, they lose the ability to shift their body's gravity center and cannot keep posture balance. Sitting rehabilitation is the basis for stroke patients to recover the stand capability. Only the stroke patients can sit stably, can they practice standing.

The purpose of static standing rehabilitation is to reestablish a stable standing posture and standing balance ability for the stroke patients by practicing the standing balance ability. Since standing balance is the basis of safe walking, the successful reestablishment of the standing balance ability enables the stroke patients to maintain the stability of the trunk when they are in a standing posture, and other limbs of the stroke patients can move freely and the body's gravity center can be shifted to perform various functional activities in the standing posture.

All the time, the evaluation of the rehabilitation performance of the stroke patients, especially the most basic and the most important performances of the standing posture, the sitting posture and the posture changes under a lying posture, will be evaluated by specific scales. When using this type of evaluation scale for evaluation, the routine method of rehabilitation physicians, physical therapists, occupational therapists or rehabilitation nurses is to judge and evaluate the performance based on their own understanding of the disease symptoms of the patient and their experiences in treatment and rehabilitation. Generally speaking, most of the evaluations performed by medical staff for the postural maintenance of the patient's trunk or limb are qualitative assessment results, rather than quantitative assessment results.

In addition, the stroke patients need a rehabilitation training program according to the evaluation results so that they can gradually recover their sitting and standing abilities. Through the rehabilitation training program, the patients can strengthen the strength, coordination and durability of their muscles, generate anti-gravity movements, such as standing, raising hands, etc., and regain balance skills, so as to maintain the body's gravity center without imbalance in sitting and standing postures.

In order to provide qualitative and quantitative evaluation results for the rehabilitation performances of the stroke patients, and to set the rehabilitation training program based on the evaluation results, a more accurate evaluation for evaluating rehabilitation effectiveness is needed to provide the stroke patients with more appropriate treatment methods and training programs.

### SUMMARY OF THE INVENTION

In order to complement the conventional qualitative rehabilitation assessment, the present invention provides an evaluation device and method, which uses motion sensors to sense posture changes of trunk and limbs of stroke patients in a posture such as a standing posture, a sitting posture, a lying posture and a laying posture, and analyzes quantitatively rehabilitative effects of the patients in exercising the standing posture, the sitting posture and the lying posture based on the sensed motion data.

One aspect of the present invention is to provide a device for evaluating a postural control ability of a subject, which includes: a sensing unit, a database for storing a reference value of posture maintenance of each body part under a specific one of a plurality of referencing body postures, and a processor, wherein the sensing unit includes a plurality of motion sensors correspondingly configured on a plurality of body parts of the subject to generate a plurality of sensing data, wherein each of the plurality of sensing data includes static sensing data and dynamic sensing data, the processor is configured to receive the plurality of sensing data from the sensing unit and the reference value of posture maintenance from the database. The processor includes a first operating unit of posture determination and a second operating unit of evaluating the postural control ability, wherein the first operating unit of posture determination is configured to form a body posture configuration according to the plurality of static sensing data, so as to determine a specific static body posture of the subject, wherein the specific static body posture corresponds to the specific one of the plurality of referencing body postures, and the second operating unit of evaluating the postural control ability is configured to process the plurality of dynamic sensing data to obtain a posture maintenance parameter for a specific one of the plurality of body parts, and compare the posture maintenance parameter with the corresponding reference value of posture maintenance of the specific body part under the specific static body posture, so as to generate an evaluation result of the postural control ability.

Another aspect of the present invention is to provide a method for evaluating a postural control ability of a subject, the method includes the steps of configuring a plurality of motion sensors on a plurality of body parts of the subject correspondingly to generate a plurality of sensing data, each of the plurality of sensing data including static sensing data and dynamic sensing data, performing a posture determination procedure by applying the plurality of static sensing data to determine a specific body posture of the subject, and performing an evaluation procedure to evaluate the postural control ability. The evaluation procedure includes the steps of processing each of the plurality of dynamic sensing data to obtain a posture maintenance parameter for each of the plurality of body parts under the corresponding body posture, and comparing the posture maintenance parameter with a reference value of posture maintenance for each of the body parts under the specific body posture so as to generate an evaluation result of the postural control ability.

A further aspect of the present invention is to provide a method for evaluating a postural control ability of a subject, the method includes the steps of configuring a plurality of motion sensors on a plurality of body parts of the subject correspondingly to generate a plurality of dynamic sensing data, and performing an evaluation procedure for the postural control ability under a specific body posture, wherein the evaluation procedure includes the steps of processing each of the plurality of dynamic sensing data to obtain a posture maintenance parameter for each of the plurality of body parts under the specific body posture, and comparing the posture maintenance parameter with a reference value of posture maintenance for each of the body parts under the specific body posture, so as to generate an evaluation result of the postural control ability.

Other objective, advantages and efficacies of the present invention will be described in detail below taken from the preferred embodiments with reference to the accompanying drawings.

### BRIEF DESCRIPTION OF THE DRAWINGS

The objectives and advantages of the present invention will become more readily apparent to those ordinarily skilled in the art after reviewing the following detailed descriptions and accompanying drawings.

FIG. 1 is a schematic diagram of the postural control evaluating device of the present invention.

FIG. 2A is a diagram showing the arrangement of the motion sensors on the subject with the sitting posture and the measured acceleration values or angular values.

FIG. 2B is a diagram showing the arrangement of the motion sensors on the subject with the standing posture and the measured acceleration values or angular values.

FIG. 3 is a flow chart of the postural control evaluating method of the present invention.

FIG. 4A is a side view showing the definition of a sitting posture configuration.

FIG. 4B is a front view showing the definition of the sitting posture configuration.

FIG. 5A is a side view showing the definition of an assisted standing posture configuration.

FIG. 5B is a front view showing the definition of the assisted standing posture configuration.

FIG. 6A is a side view showing the definition of a standing posture configuration.

FIG. 6B is a front view showing the definition of the standing posture configuration.

FIG. 7A is a side view showing the definition of a standing on one foot posture configuration with left foot.

FIG. 7B is a front view showing the definition of the standing on one foot posture configuration with left foot.

FIG. 8A is a side view showing the definition of a standing on one foot posture configuration with right foot.

FIG. 8B is a front view showing the definition of the standing on one foot posture configuration with right foot.

FIG. 9A is a top view showing the definition of a lying posture configuration.

FIG. 9B is a side view showing the definition of the lying posture configuration.

FIG. 10A is a diagram showing the definition of a left lying posture configuration.

FIG. 10B is a diagram showing the definition of a right lying posture configuration.

FIG. 11A is a diagram showing evaluation for rehabilitation effect of the sitting posture.

FIG. 11B is a diagram showing evaluation for rehabilitation effect of the standing posture.

### DETAILED DESCRIPTION OF THE PREFERRED EMBODIMENTS

The present invention will now be described more specifically with reference to the following embodiments. It is to be noted that the following descriptions of the preferred embodiments of this invention are presented herein for purpose of illustration and description only; they are not intended to be exhaustive or to be limited to the precise form disclosed.

The foregoing and other technical content, features and effects of the present invention will be clearly presented in the detailed descriptions of multiple embodiments below with reference to the drawings. Although the stroke patients are illustrated as subjects of the posture control evaluating device of the present invention in the embodiments of the present disclosure, the posture control evaluating device of the present invention is suitable for any subject that needs to be evaluated. Therefore, the term “subject” used in this disclosure includes, but is not limited to, healthy subjects, patients, injured subjects or disabled persons.

Regarding the rehabilitation status of the stroke patients, the balance and mobility of the stroke patients are generally assessed according to Table 1 “The Postural Assessment Scale For Stroke Patients”, which includes the ability of maintaining the body postures, such as sitting, standing, lying, etc., and the ability to interchange the posture among these different body postures.

The term “evaluation content” used in the present invention refers to a scale used to evaluate the posture control ability of a subject, especially a scale used to evaluate the posture control ability of stroke patients. In addition to Table 1 “The Postural Assessment Scale For Stroke Patients”, the evaluation content also includes other assessment forms, for example, Item 5 (upper limbs mobility) and Item 6 (lower limbs mobility) of Table 2 “National Institutes of Health Stroke Scale (NIHSS)”.

TABLE 1
The Postural Assessment Scale For Stroke Patients Evaluation postures:
The balance and basic mobility
A. Maintaining Posture

| | | | | |
|---|---|---|---|---|
| 1. Sitting without | 0 □ | 1 □ | 2 □ | 3 □ |
| support | Cannot sit | Can sit with | Can sit for | Can sit for |
| (Have subject sit | | slight support | more than 10 | more than 5 |
| on bench/mat | | | seconds | minutes |
| without back | | | without support | without support |
| support with feet | | | | |
| flat on floor.) | | | | |
| 2. Standing with | 0 □ | 1 □ | 2 □ | 3 □ |
| support | Cannot stand, | Can stand with | Can stand with | Can stand with |
| (Have subject stand, | even with | strong | moderate | support |
| providing support as | support | support of | support of | on only |
| needed. Evaluate | | two persons | one person | one hand |
| only the ability to | | | | |
| stand with or without | | | | |
| support. Do not | | | | |
| consider the quality | | | | |
| of stance.) | | | | |
| 3. Standing without | 0 □ | 1 □ | 2 □ | 3 □ |
| support | Cannot stand | Can stand | Can stand | Can stand |
| (Have subject | without | without | without support | without support |
| stand without | support | support for | for more than 1 | for more than 1 |
| support. Evaluate | | more than 10 | minute or | minute and |
| only the ability to | | seconds or | stands slightly | perform arm |
| stand with or | | leans heavily | asymmetrically | movements |
| without support. | | on one leg | | above the |
| Do not consider | | | | shoulder at the |
| quality of stance.) | | | | same time |
| 4. Standing on | 0 □ | 1 □ | 2 □ | 3 □ |
| nonparetic leg (or | Cannot stand | Can stand for | Can stand for | Can stand for |
| left leg) | | a few | more than 5 | more than 10 |
| | | seconds | seconds | seconds |
| 5. Standing on | 0 □ | 1 □ | 2 □ | 3 □ |
| paretic leg (or right | Cannot stand | Can stand for | Can stand for | Can stand for |
| leg) | | a few | more than 5 | more than 10 |
| | | seconds | seconds | seconds |

Total Score of Items 1~5:
B. Changing Posture
(Items 6 to 11 are performed when the patient lies or sits on a 50 cm high table, and Items
9 to 12 should be performed only when the patient can sit or stand alone. )

| | | Can | Can | Can |
|---|---|---|---|---|
| | Cannot | perform | perform | perform |
| | perform | the activity | the activity | the activity |
| | the | with much | with a little | without |
| | activity | help | help | help |
| 6. Supine posture to | 0 □ | 1 □ | 2 □ | 3 □ |
| paretic side lateral | | | | |
| (or left side) | | | | |
| 7. Supine posture to | 0 □ | 1 □ | 2 □ | 3 □ |
| non-paretic side | | | | |
| lateral (or right | | | | |
| side) | | | | |
| 8. Supine posture to | 0 □ | 1 □ | 2 □ | 3 □ |
| sitting up | | | | |
| 9. Sitting to supine | 0 □ | 1 □ | 2 □ | 3 □ |
| posture | | | | |
| 10. Sitting to | 0 □ | 1 □ | 2 □ | 3 □ |
| standing up | | | | |
| 11. Standing up | 0 □ | 1 □ | 2 □ | 3 □ |
| to sitting down | | | | |
| 12. standing and | 0 □ | 1 □ | 2 □ | 3 □ |
| picking up a pencil | | | | |
| on the floor | | | | |

Total Scores of Items 6~12:
Total Scores of Items 1~12:

TABLE 2
National Institutes of Health (NIH) Stroke Scale
Time

| 1a. Level of | Alert 0 | |
|---|---|---|
| consciousness | Drowsy 1 | |
| | Stuporous 2 | |
| | Coma 3 | |
| 1b. Level of | Answers both questions | |
| consciousness | correctly 0 | |
| (Ask patient's | Answers one question | |
| month and | correctly 1 | |
| age) | Answers neither question | |
| | correctly 2 | |
| 1c. Level of | Performs both tasks | |
| consciousness | correctly 0 | |
| (Ask patient to | Performs one task | |
| open/close | correctly 1 | |
| eyes, grip and release | Performs neither task | |
| unaffected hand.) | correctly 2 | |
| 2. Best gaze | Normal 0 | |
| (Horizontal extraocular | Partial gaze palsy 1 | |
| movements by voluntary or | Forced deviation 2 | |
| reflexive testing). | | |
| 3. Visual fields (left, right, | No visual loss 0 | |
| upper and lower quadrants | Partial hemianopia 1 | |
| are tested) | Complete hemianopia 2 | |
| 4. Facial Palsy (Ask the | Normal 0 | |
| patient to show teeth and | Asymmetry on smiling 1 | |
| close eyes ) | Total or near-total | |
| | paralysis of lower face 2 | |
| | Absence of facial | |
| | movement in the upper | |
| | and lower face 3 | |
| 5. Upper limbs movements | No drift 0 | Left |
| (The patient closes his eyes | Drift 1 | (5a) |
| and extends his arms at 90 | Some effort | Right |
| degrees (if sitting) or 45 | against gravity 2 | (5b) |
| degrees (if supine) for 10 | No effort | |
| seconds.) | against gravity 3 | |
| | No movement 4 | |
| 6. Lower limbs movements | No drift 0 | Left |
| (The patient lies and holds a | Drift 1 | (6a) |
| leg at 30 degrees for 5 | Some effort | Right |
| seconds.) | against gravity 2 | (6b) |
| | No effort | |
| | against gravity 3 | |
| | No movement 4 | |

| | |
|---|---|
| 7. Limb ataxia | No ataxia 0 |
| | Ataxia in one limb 1 |
| | Ataxia in two limbs 2 |
| 8. Sensory (face, trunk, both | Normal 0 |
| arms and both legs are | Partial sensory loss 1 |
| tested) | Complete sensory loss |
| | (Coma) 2 |
| 9. Language (Describe the | No aphasia 0 |
| scene to obtain the level of | Aphasia in any item 1 |
| speaking fluency, naming | Severe aphasia 2 |
| the items, reading the | Mute/global aphasia 3 |
| sentences and | |
| understanding) | |
| 10. Dysarthria (slurred) | Normal 0 |
| | Can be understood |
| | partially 1 |
| | Cannot be understood 2 |
| 11. Neglect (Any one of | No abnormality 0 |
| visual sense, tactile, | Visual, tactile, auditory, |
| auditory, spatial sense, and | spatial, or personal |
| attention to the stimulus | inattention 1 |
| disappears) | Profound hemi-inattention |
| | or extinction to two or |
| | more modality 2 |

Total Score (0-42)

When the medical personnel, such as rehabilitation physicians, physical therapists, rehabilitation nurses, etc., evaluates rehabilitation status for the stroke patients based on “The Postural Assessment Scale for Stroke Patients”, they will visually judge which posture (i.e, the evaluation items such as sitting, standing or other body postures) in this assessment scale that the patient at, and the evaluation conditions in these evaluation items include the length of time that the body posture can be maintained, and the level of assistance required. As for the effectiveness of the body posture maintenance, or whether the performance of the posture maintenance is qualified, it is evaluated in a qualitative way based on the subjective cognition of the medical personnel such as physical therapists.

Compared with the above-mentioned conventional method for evaluating the rehabilitation status of the stroke patients operated by the medical personnel, the technical feature of the present invention is to use a motion sensing device. According to the evaluation process of “The Postural Assessment Scale for Stroke Patients”, the motion sensing device senses the current body posture of the stroke patient first, and automatically and correspondingly selects the evaluation items, including a plurality sitting evaluation items and a plurality standing evaluation items, to be evaluated in “The Postural Assessment Scale for Stroke Patients” based on the sensed body posture. After the evaluation item is selected, the motion sensing device senses the changes in the selected posture over time (for example, the physical quantities such as deflection angle, and jitter or shaking amplitude or frequency of the trunk or limbs, i.e. a limb motion performed by no intention of the subject, an unintended or non-autonomous limb motion or behavior of the subject), and calculates these physical quantities to evaluate whether the effectiveness of the body posture maintenance meets a posture maintenance reference value in a quantitative way, so as to objectively determine the maintenance time that meets the posture maintenance reference value. That is, the present invention quantifies the posture change to evaluate the rehabilitation effect, and uses the objective data to determine whether the posture change meets a threshold value. By using the method above, the present invention can be applied to the judgment of the maintenance time in “The Postural Assessment Scale for Stroke Patients”, so that the evaluation of the rehabilitation effect for the stroke patients is more objective and accurate.

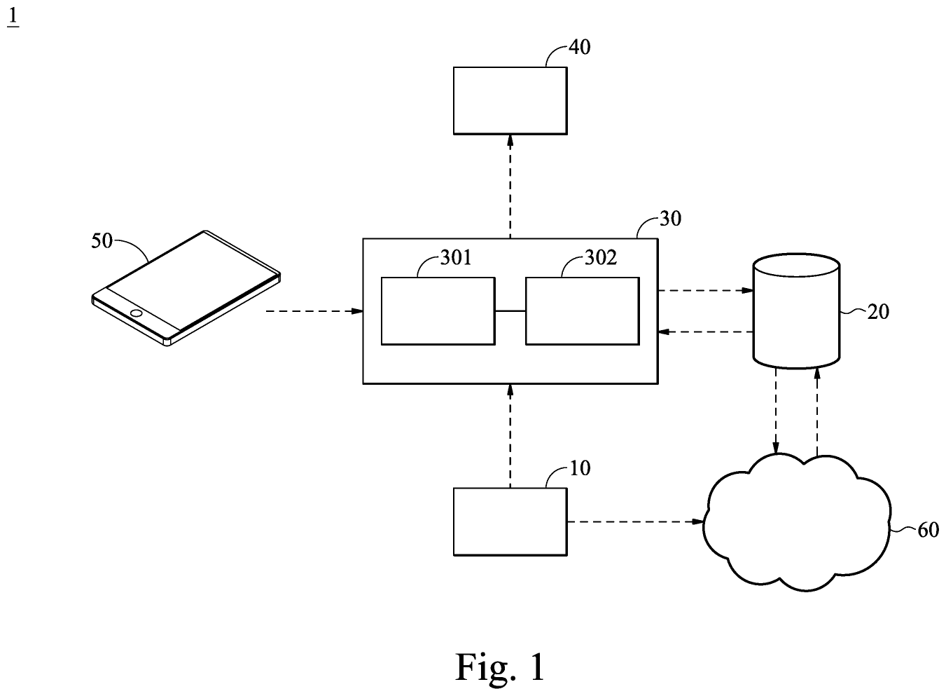

FIG. 1 is a schematic diagram of the posture control evaluating device 1 of the present invention. As shown in FIG. 1, the posture control evaluating device 1 of the present invention includes a sensing unit 10, a database 20, a processor 30, a display 40 and an input device 50. The sensing unit 10 includes a plurality of motion sensors, and these motion sensors are correspondingly configured on a plurality of body parts of a subject (such as trunk, upper arms, wrists, thighs, and lower calves) to generate a plurality of sensing data. The database 20 is used to store reference values of posture maintenance of each body part under a plurality of referencing body postures. For example, the reference value of posture maintenance may be a reference maintenance time, a reference deflection angle, a reference jitter time, a reference jitter amplitude and a reference jitter frequency of each body part in the sitting posture. The reference value of posture maintenance may also be a reference maintenance time, a reference deflection angle, a reference jitter time, a reference jitter amplitude and a reference jitter frequency of each body part in the standing posture. The processor 30 receives the plurality of sensing data from the sensing unit 10 and the reference value of posture maintenance from the database 20.

The processor 30 includes an operating unit of posture determination 301 and an operating unit of evaluating the postural control ability 302. When the subject is in a static state, the motion sensors of the sensing unit 10 measure the angle or azimuth angle of each body part relative to a reference direction, and obtain static sensing data. The operating unit of posture determination 301 determines that the subject is in the standing, sitting, squatting, prone or lying posture (hereinafter referred to as a specific static body posture) according to the static sensing data of each body part. When the specific static body posture of the subject persists, some of the subject's unintended or uncontrollable movements such as jitter, shaking or tilting may occur at various body parts, and motion sensors 101 of the sensing unit 10 measure dynamic sensing data such as acceleration, an angular velocity, a displacement and a frequency of the movements. The operating unit of evaluating the postural control ability 302 processes the dynamic sensing data of each body part to obtain posture maintenance parameters such as the maintenance time, the deflection angle, the jitter time, the jitter amplitude and the jitter frequency of each body part, and compares each posture maintenance parameter with the corresponding posture maintenance reference value of each body part under the determined specific static body posture, to produce an evaluation result of the posture control ability.

The display 40 is coupled to the processor 30 for displaying the evaluation result of the posture control ability. The input device 50 may be a device with an input interface such as a computer, a mobile phone, or a tablet, which is connected to the processor 30 electrically or wirelessly, and is used to receive an input from an operator, such as a medical staff or a patient's helper, and control the processor 30 through the input interface. The operator may initiate the sensing process and calculation process of the processor 30 through an application program (APP) in the mobile phone or the tablet or a software program in the computer. The evaluation result of the posture control ability generated by the processor 30 can be stored in the database 20 or a cloud database 60. The cloud database 60 can receive the evaluation result of the posture control ability from the processor 30 directly, or receive the evaluation result of the posture control ability stored in the database 20. The cloud database 60 can also be used to store the sensing data from the sensing unit 10 and then provide the sensing data to the processor 30.

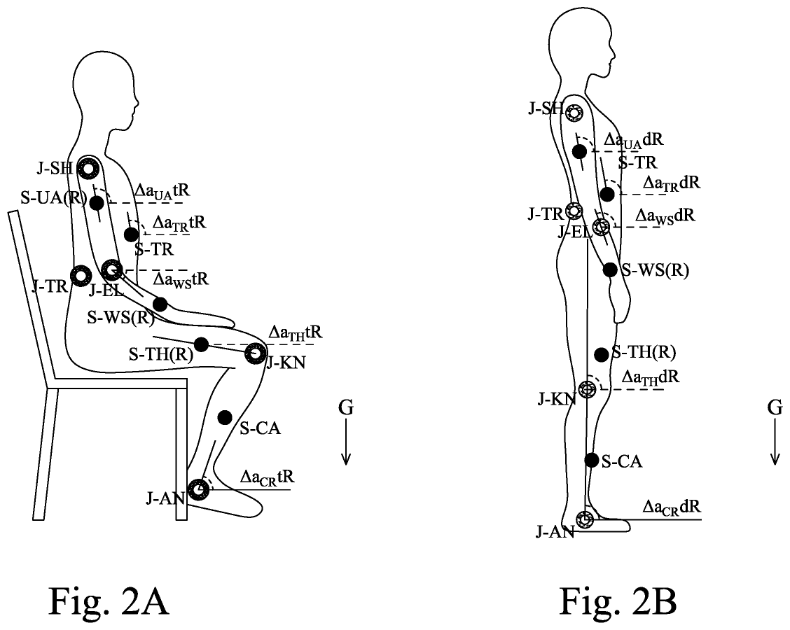

Hereinafter, the configuration of the motion sensors of the posture control evaluating device 1 and a posture sensing method of the present invention will be further described. Please refer to FIG. 2A and FIG. 2B, which are arrangements of the motion sensors on the subject with the sitting and standing postures, respectively. In FIG. 2A and FIG. 2B, J-TR, J-SH, J-EL, J-KN and J-AN represent the trunk, shoulder, elbow, knee and ankle joints, respectively, and S-UA(R), S-TR, S-WS(R), S-TH(R) and S-CA(R) represent the motion sensors configured on the right upper arm, the upper trunk, the right wrist, the right thigh and the right calf. Although only the motion sensors on the right side are shown in FIGS. 2A and 2B, the motion sensors of the present invention can also be symmetrically configured on the left upper arm, the left wrist, the left thigh and the left calf. According to one embodiment of the present invention, the posture control evaluating device 1 may have 9 motion sensors (i.e. one on the upper trunk, four on the right side and four on the left side). However, the number of the motion sensors can be increased or decreased depending on the needs of the evaluation. For example, in order to evaluate the posture control of the affected or the Paretic side of the stroke patient, the motion sensors can only be configured on the upper trunk and the affected or the Paretic side of the stroke patient. Optionally, the sensing unit 10 of the posture control evaluating device 1 further includes a support sensor 102 (as shown in FIG. 1), such as a contact sensor, configured on the limb of the stroke patient. When the stroke patient is supported by a supporter, the contact sensor 102 can sense the supporter's touch or contact so as to make the contact sensor 102 send out a signal.

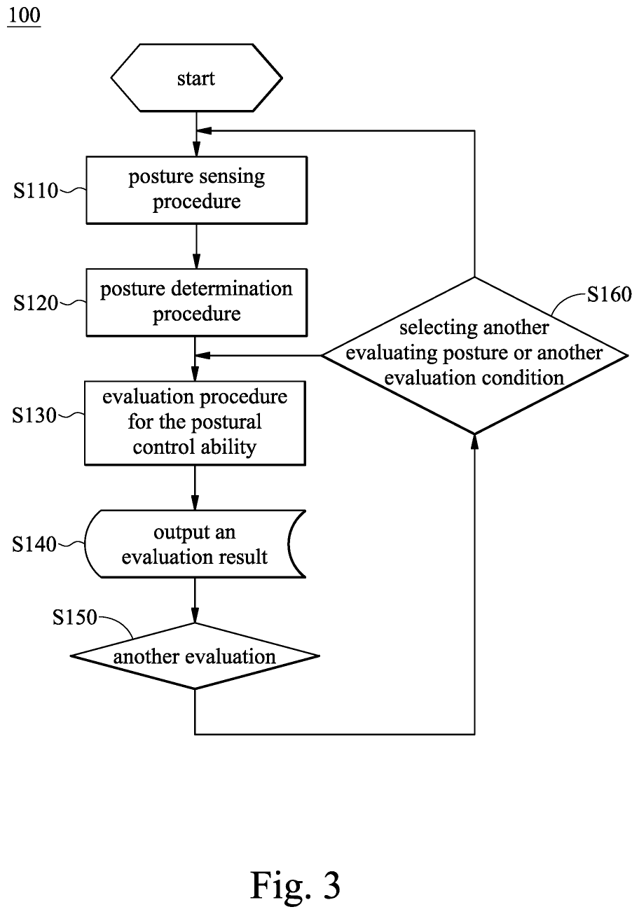

In another aspect, the present invention provides a method for evaluating a postural control ability of a subject. FIG. 3 is a flow chart of the postural control evaluating method 100 of the present invention. As mentioned above, when an operator activates the processor 30 of the posture control evaluating device 1 as shown in FIG. 1, a posture sensing procedure S110 is initiated.

In S110, the static sensing data of the trunk and each limb can be obtained using the motion sensors arranged on the trunk and each limb as shown in FIGS. 2A and 2B. That is, the angle or azimuth angle of the trunk and each limb relate to a reference direction. In the present invention, the reference direction may be a horizontal direction or a gravity direction. In a posture determination procedure S120 of the present invention, the operating unit of posture determination 301 as shown in FIG. 1 is used to transform the static sensing data of the trunk and each limb into a plurality of vectors, and the following mathematical matrix or the vector model is used to present a function of the body posture configuration for indicating the body posture configuration:

[Trunk (TR); Right upper arm (UpAR); Right forearm-wrist (FrA-WsR); Right thigh (ThighR); Right calf (CalfR); Left upper arm (UpAL); Left forearm-wrist (FrA-WsL); Left thigh (ThighL); Left calf (CalfL)].

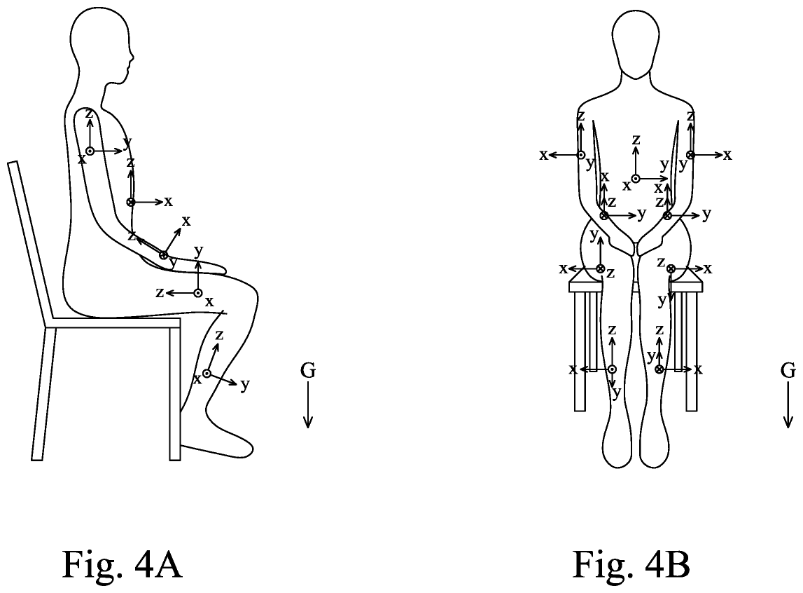

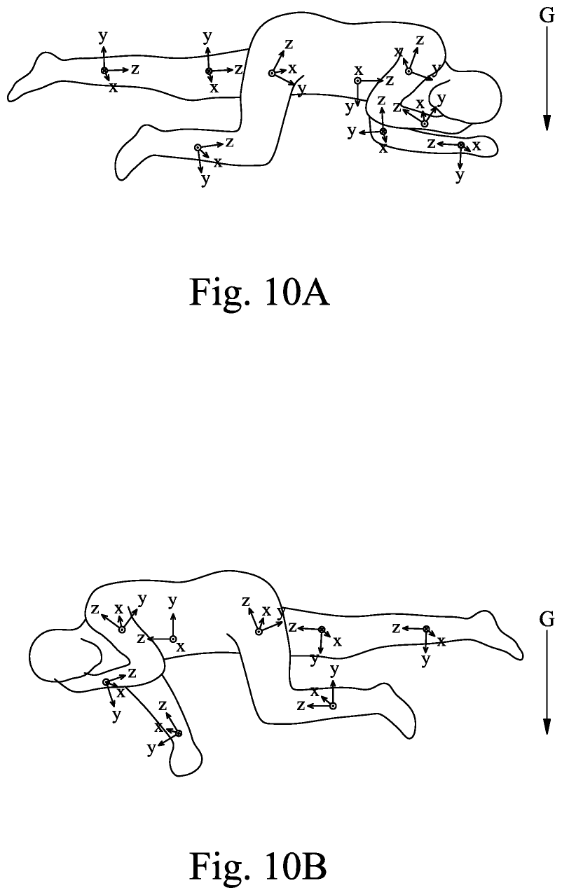

FIGS. 4A to 10B are the definitions of various body posture configurations. As shown in the drawings, each body posture configuration is a function of each body posture configuration, which is a vector representation associated with the angles or the orientations of various body parts in the static state related to the x, y and z directions of a reference coordinate, correspondingly, and each body posture configuration, i.e., its corresponding function of the body posture configuration, has its own characteristic value. The following are the characteristic values of each body posture configuration in FIGS. 4A to 10B:

the characteristic value for the sitting posture configuration is [TR: 0,0,−1g; UpAR: 0,0,−1g; FrA-WsR: −gx,0,−gz; ThighR: 0,−1g, 0; CalfR: 0,gy,−gz; UpAL: 0,0,−1g; FrA-WsL: −gx,0,−gz; ThighL: 0,1g,0; CalfL: 0,−gy,−gz];
the characteristic value for the posture configuration of standing with support is: [TR: 0,0,−1g; UpAR: −gx,−gy,−gz; FrA-WsR: −0,−gy,−gz; ThighR: 0,0,−1g; CalfR: 0,0,−1g; UpAL: −gx, gy,−gz; FrA-WsL: 0,gy,−gz; ThighL: 0,0,−1g; CalfL: 0,0,−1g];
the characteristic value for the standing posture configuration is [TR: 0,0,−1g; UpAR: 0,0,−1g; FrA-WsR: 0,0,−1g; ThighR: 0,0,−1g; CalfR: 0,0,−1g; UpAL: 0,0,−1g; FrA-WsL: 0,0,−1g; ThighL: 0,0,−1g; CalfL: 0,0,−1g];
the characteristic value for the posture configuration of standing on the left leg is: [TR: 0,0,−1g; UpAR: 0,0,−1g; FrA-WsR: 0,−gy,−gz (or gx, gy, gz); ThighR: 0,−gy,−gz; CalfR: 0,0,−1g; UpAL: 0, 0,−1g; FrA-WsL: 0,0,−1g (or gx, gy, gz); ThighL: 0,0,−1g; CalfL: 0,0,−1g];
the characteristic value for the posture configuration of standing on the right leg is: [TR: 0,0,−1g; UpAR: 0,0,−1g; FrA-WsR: 0,0,−1g (or gx, gy, gz); ThighR: 0,0,−1g; CalfR: 0,0,−1g; UpAL: 0, 0,−1g; FrA-WsL: 0,gy,−gz (or gx, gy, gz); ThighL: 0,gy,−gz; CalfL: 0,0,−1g];
the characteristic value for the lying posture configuration is: [TR: −1g,0,0; UpAR: 0,−1g,0; FrA-WsR: 0,−1g,0; ThighR: 0,−1g,0; CalfR: 0,−1g,0; UpAL: 0,1g,0; FrA-WsL: 0,1g,0; ThighL: 0,1g,0; CalfL: 0,1g,0];
the characteristic value for the left lying posture configuration is: [TR: 0,1g,0; UpAR: −gx,gy,−gz; FrA-WsR: −gx,−gy,−gz; ThighR: −gx,gy,−gz; CalfR: gx,gy˜1g,−gz˜−0; UpAL: gx,gy,−gz; FrA-WsL: gx,gy˜1g,gz˜0; ThighL: gx,−gy,−gz; CalfL: gx,−gy,−gz˜−0];
the characteristic value for the right lying posture configuration is: [TR: 0,−1g,0; UpAR: gx,gy,−gz; FrA-WsR: −gx,gy,−gz; ThighR: gx-0,gy˜1g,−gz˜−0; CalfR: −gx˜−0,−gy˜−1g,gz˜0; UpAL: −gx,−gy,−gz; FrA-WsL: gx,gy,−gz; ThighL: −gx,−gy−gz; CalfL: −gx˜−0,−gy˜−1g,−gz˜−0].

Please refer to FIG. 3, in the posture determination procedure S120 of the present invention, the operating unit of posture determination 301 further compares the body posture configuration to the various body posture configurations and their characteristic values shown in FIGS. 4A to 10B, to determine the specific body posture of the subject. This process is equivalent to the step of determining the evaluation items of the stroke patient among Items 1 to 12 in Table 1 “The Posture Assessment Scale for Stroke Patients” when the evaluation is performed using Table 1.

In the posture sensing procedure S110, the motion sensors can obtain the dynamic sensing data of the trunk and each limb. When the specific static body posture of the subject persists, an ideal state is to maintain this specific static body posture. However, the subject may generate unintended movements such as jitter, shaking or tilting under this specific static body posture, and the specific static body posture would be changed to a dynamic body posture because the maintenance of the specific static body posture may be interfered by the unintended dynamic motions. At this time, the motion sensors of the sensing unit 10 measure the dynamic sensing data such as the acceleration, the angular velocity, the displacement and the frequency of the unintended movement occurred at each body part. Preferably, the dynamic sensing data includes at least two, at least three, or all parameters such as the acceleration, the angular velocity, the displacement and the frequency. The parameters related to the dynamic sensing data are described as follows:

Δa_{UA}tR: when a sitting posture persists, an unintended movement such as jitter, shaking or tilting occurred at (right) upper arm is represented as an acceleration or an angular change relative to the gravity direction;
Δa_{TR}tR: when a sitting posture persists, an unintended movement such as jitter, shaking or tilting occurred at the trunk is represented as an acceleration or an angular change relative to the gravity direction;
Δa_{WS}tR: when a sitting posture persists, an unintended movement such as jitter, shaking or tilting occurred at (right) wrist is represented as an acceleration or an angular change relative to the gravity direction;
Δa_{TH}tR: when a sitting posture persists, an unintended movement such as jitter, shaking or tilting occurred at (right) thigh is represented as an acceleration or an angular change relative to the gravity direction;
Δa_{CA}tR: when a sitting posture persists, an unintended movement such as jitter, shaking or tilting occurred at (right) calf is represented as an acceleration or an angular change relative to the gravity direction;
Δa_{UA}dR: when a standing posture persists, an unintended movement such as jitter, shaking or tilting occurred at (right) upper arm is represented as an acceleration or an angular change relative to the gravity direction;
Δa_{TR}dR: when a standing posture persists, an unintended movement such as jitter, shaking or tilting occurred at the trunk is represented as an acceleration or an angular change relative to the gravity direction;
Δa_{WS}dR: when a standing posture persists, an unintended movement such as jitter, shaking or tilting occurred at (right) wrist is represented as an acceleration or an angular change relative to the gravity direction;
Δa_{TH}dR: when a standing posture persists, an unintended movement such as jitter, shaking or tilting occurred at (right) thigh is represented as an acceleration or an angular change relative to the gravity direction;
Δa_{CA}dR: when a standing posture persists, an unintended movement such as jitter, shaking or tilting occurred at (right) calf is represented as an acceleration or an angular change relative to the gravity direction.

After the specific static body posture of the subject is determined in the posture determination procedure S120, the method enters an evaluation procedure for the postural control ability S130. In the evaluation procedure for the postural control ability S130, the operating unit of evaluating the postural control ability 302 as shown in FIG. 1 is used to process the measured dynamic sensing data to obtain posture maintenance parameters of various body parts including the deflection angle, jitter amplitude, jitter frequency and jitter time of the trunk and each limb, and compare the posture maintenance parameters to the standard data of the same body part under the same body posture. That is, the deflection angle, the jitter amplitude, the jitter frequency, and the jitter time are compared with the entire time of maintaining the body posture, to determine whether the posture maintenance status meets the criteria for a specific evaluation item. This process is equivalent to the step of determining the maintenance ability or the level of assistance requirement of the stroke patient in one of the evaluation items based on the evaluation criterion of 0 to 3 points in Table 1 “The Posture Assessment Scale for Stroke Patients” when the evaluation is performed using Table 1.

In the evaluation procedure for the postural control ability S130, the unintended movements such as jitter, shaking or tilting are relatively easy to occur in some body parts (e.g. the trunk, the wrist, the thigh and the calf), and thus the trunk, the wrist, the thigh and the calf are suitable for conducting the posture maintenance evaluation. The exemplary equation is as follows, where F_{BD_RhP} represents the function of the rehabilitation effect of each body part, F_{BD_RhM} represents the function of the rehabilitation measurement of each body part, TH represents the standard data, I is an Identity matrix, THxx, Thyy and THzz represent the standard data related to x, y and z axis directions of the reference coordinates, respectively. BD(t) represents the function of posture changes of the upper body of the trunk, wrist, thigh or calf with time, including ax_BD(t), ay_BD(t) and az_BD(t) representing the acceleration components of posture changes, respectively related to the x, y, and z directions of the reference coordinate, or the angular changes relative to the Gravity.

$$\begin{matrix}{F}_{BD\_RhP} = I \cdot TH - {F}_{BD\_RhM} \\ = [ \begin{matrix}TH xx - ax\_BD ( t ) & 0 & 0 \\ 0 & TH yy - ay\_BD ( t ) & 0 \\ 0 & 0 & TH zz - az\_BD ( t )\end{matrix} ]\end{matrix} \qquad Equation ( 1 )$$

The result of Equation (1) is substituted into Equation (2) to obtain an evaluation result of the rehabilitation effect, and the evaluation result is output in S140.

AF_{BD_RhP}=det(F_{BD_RhP}) Equation (2)

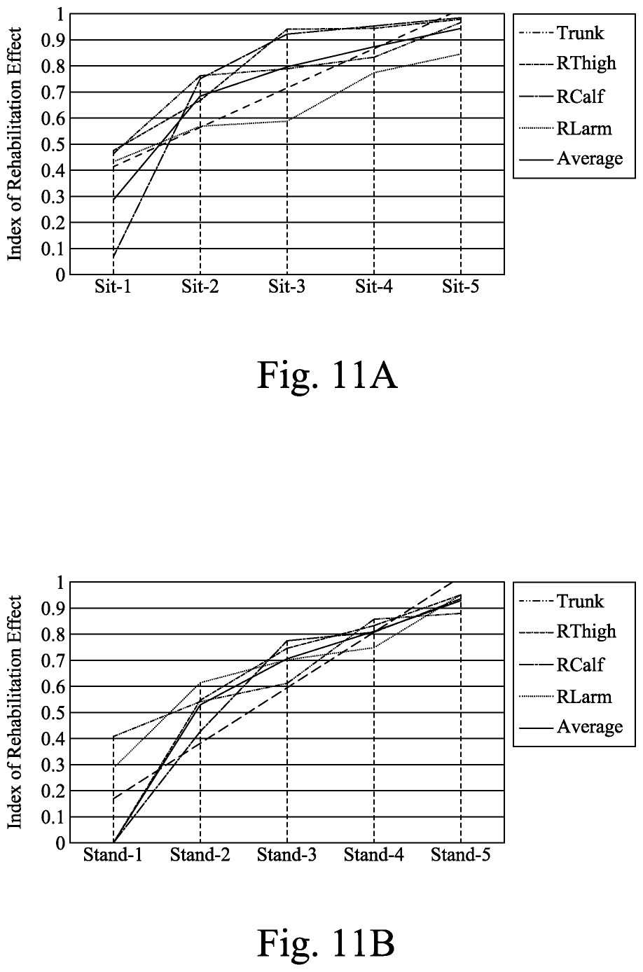

As for the index of rehabilitation effect generated through the above equations, the indexes of the progression and evaluation for rehabilitation effect in the sitting posture and the progression and evaluation for rehabilitation effect in the standing posture are respectively shown in FIG. 11A and FIG. 11B. In FIG. 11A, a stroke patient, a non-stroke patient, or an unspecified operator engaged in the sitting rehabilitation, the rehabilitation effects of the trunk, the right forearm, the right thigh and the right calf in the sitting posture were evaluated 5 times, namely, Sit-1, Sit-2, Sit-3, Sit-4 and Sit-5, respectively. It can be seen from these 5 evaluations that the index of rehabilitation effect (the average of the four body parts) is improved from 0.29 at Sit-1 (trunk: 0.47, right forearm: 0.43, right thigh: 0.48, right calf: 0.07) to 0.69 at Sit-2 (trunk: 0.77, right forearm: 0.58, right thigh: 0.67, right calf: 0.75), 0.8 at Sit-3 (trunk: 0.79, right forearm: 0.8, right thigh: 0.95, right calf: 0.92), 0.87 at Sit-4 (trunk: 0.83, right forearm: 0.78, right thigh: 0.94, right calf: 0.78) and 0.95 at Sit-5 (trunk: 0.97, right forearm: 0.85, right thigh: 0.98, right calf: 0.98). It can be seen from the progress of the posture rehabilitation effect that the maintenance ability in the sitting posture of the stroke patient gradually returns to the complete recovery state in which the rehabilitation effect index is 1. In this recovery state, the tremor, the jitter, the shaking or the deflection of the trunk and each limb are restored to a normal healthy state.

Similarly, FIG. 11B is a diagram showing evaluation for rehabilitation effect in the standing posture. For an unspecified stroke patient or other patient, or an unspecified operator doing the rehabilitation at the posture of standing without support, the rehabilitation effects of the trunk, the right forearm, the right thigh and the right calf in the standing posture were evaluated for 5 times, namely, Stand-1, Stand-2, Stand-3, Stand-4 and Stand-5, respectively. It can be seen from these 5 evaluations that the index of rehabilitation effect is improved from 0 at Stand-1 (trunk: 0.28, right forearm: 0.41, right thigh: 0, right calf: 0) to 0.53 at Stand-2 (trunk: 0.62, right forearm: 0.54, right thigh: 0.55, right calf: 0.42), 0.71 at Stand-3 (trunk: 0.71, right forearm: 0.62, right thigh: 0.76, right calf: 0.78), 0.82 at Stand-4 (trunk: 0.76, right forearm: 0.87, right thigh: 0.85, right calf: 0.82) and 0.95 at Stand-5 (trunk: 0.95, right forearm: 0.90, right thigh: 0.96, right calf: 0.95). It can be seen from the progress of the posture rehabilitation effect that the maintenance ability in the standing posture of the stroke patient gradually returns to the complete recovery state in which the index of rehabilitation effect is 1 from the amplitude of the unintended jitter, shaking or deflection exceeding a predetermined value or the patient cannot stand in which the rehabilitation effect index is 0). In this recovery state, the tremor, the jitter, the shaking or the deflection of the trunk and each limb are alleviated and the body of the patient is restored to a normal healthy state.

During the rehabilitation process, since the body posture maintenance ability is still in recovering, the indexes of rehabilitation effect, such as Sit-4 in FIG. 11A or Stand-4 in FIG. 11B can be used as the reference value of the posture maintenance. That is, the corresponding tremor, jitter, shaking or deflection values of these indexes of rehabilitation effect or rehabilitation performance are relatively objective and able to be regarded as the evaluation condition for “2. Can sit for more than 10 seconds without support” under the Maintaining Posture Item A-1 in Table 1, for being used to compare with the detected data such as tremor, jitter, shaking or deflection measured in the rehabilitation effectiveness evaluation, or posture maintenance parameters. Alternatively, indexes of rehabilitation effect, such as Sit-5 in FIG. 11A or Stand-5 in FIG. 11B can be used as the reference value of posture maintenance. That is, the corresponding tremor, jitter, shaking or deflection values of these indexes of rehabilitation effect is regarded as the evaluation condition for “3. Can sit for more than 5 minutes without support” under Maintaining Posture Item A-1 Table 1, for being used to compare with detected data such as tremor, jitter, shaking or deflection measured in the rehabilitation effectiveness evaluation, or posture maintenance parameters. Of course, the tremor, jitter, shaking or deflection value corresponding to the indexes of rehabilitation effect such as Sit-5 in FIG. 11A or Stand-5 in FIG. 11B can also be regarded as the reference data for comparing the tremor, jitter, shaking or deflection in the evaluation condition for “2. Can sit for more than 10 seconds without support” under the Maintenance Posture Item A-1 in Table 1.

Please refer to FIG. 3. After the evaluation result is output, the postural control evaluating method 100 of the present invention can proceed to another evaluation S150 having higher evaluation conditions, and another evaluation posture or another evaluation condition is selected in S160 to further determine the subject's postural control ability. This step can be implemented by, for example, the processor 30 and the database 20 in FIG. 1. For example, when the subject passes the evaluation condition “1. Can stand without support for more than 10 seconds, or leans heavily on one leg” of the evaluation item A-3, the posture control evaluating device 1 of the present invention can prompt the subject or the operator whether to continue with an evaluation of the evaluation condition “2. Can stand without support for more than 1 minute or stands slightly asymmetrically” of the evaluation item A-3. Alternatively, the database 20 may be used to store an evaluation content including multiple evaluation items and multiple evaluation conditions, and the processor 30 automatically performs the evaluation of the evaluation condition 2 of the evaluation item A-3 according to the evaluation items and the evaluation conditions stored in the database 20. In another embodiment, the posture control evaluating device 1 of the present invention may prompt the subject or the operator whether to change posture so as to perform the evaluation with the evaluation condition “1. Can perform the activity with much help” of another evaluation item B-11.

If the subject chooses to perform another evaluation, the procedures of S110 to S140 of the postural control evaluating method 100 of the present invention are re-performed to obtain the evaluation result of the further evaluation. In another embodiment, without changing the evaluation condition, the procedures of S110 to S140 of the postural control evaluating method 100 of the present invention can also be re-performed to verify the previous evaluation result.

According to another embodiment of the present invention, the evaluation procedure for the postural control ability S130 of the postural control evaluating method 100 of the present invention can be directly executed under a condition that a specific static body posture is specified, and the evaluation result is output in S140. In this example, S120 can be omitted, the dynamic sensing data of each body part is sensed in S110 using the motion sensors, and S130 and S140 are performed according to the aforementioned evaluation procedure for the postural control ability.

The posture control evaluating device 1 and the postural control evaluating method 100 of the present invention can not only generate the evaluation result of the postural control ability, but also provide a suitable action practice plan for the subject according to the evaluation result. For example, based on the evaluation result, an action practice content and a time allocation corresponding to the action practice content are selected from the evaluation conditions stored in the database 20. An example of the action practice plan may include: proceeding from the evaluation item A-3 “Standing without support” to the evaluation item A-4 “Standing on the unaffected or non-paretic leg” in Table 1, and then proceeding to the evaluation item A-5 “Standing on the affected or paretic leg”. Another example of the action practice plan may include: changing from the evaluation item B-12 “standing and picking up a pencil on the floor” to the evaluation item B-11 “Standing up to sitting down”, and then changing to the evaluation item B-10 “Sitting to standing up”.

Hereinafter, the posture determination procedure S120 and the evaluation procedure for the postural control ability S130 of the postural control evaluating method 100 of the present invention will be described with specific embodiments.

### Embodiment 1. Sitting without Support

If the motion sensors detect the characteristic values of the posture configuration of sitting without support, as shown in FIG. 4A and FIG. 4B, the operating unit of posture determination will select the posture in the evaluation item A-1 “Sitting without support”, or the posture in the evaluation item B-9 “Sitting to supine posture”, or the posture in the evaluation item B-10 “Sitting to standing up” in Table 1 for the subsequent postural control ability evaluations. These evaluation items include the following evaluation conditions:

A-1. cannot sit; can sit with slight support; can sit for more than 10 seconds without support; can sit for more than 5 minutes without support;
B-9. cannot perform the activity; can perform the activity with much help; can perform the activity with a little help; can perform the activity without help; and
B-10. cannot perform the activity; can perform the activity with much help; can perform the activity with a little help; can perform the activity without help.

### Embodiment 2. Standing with Support

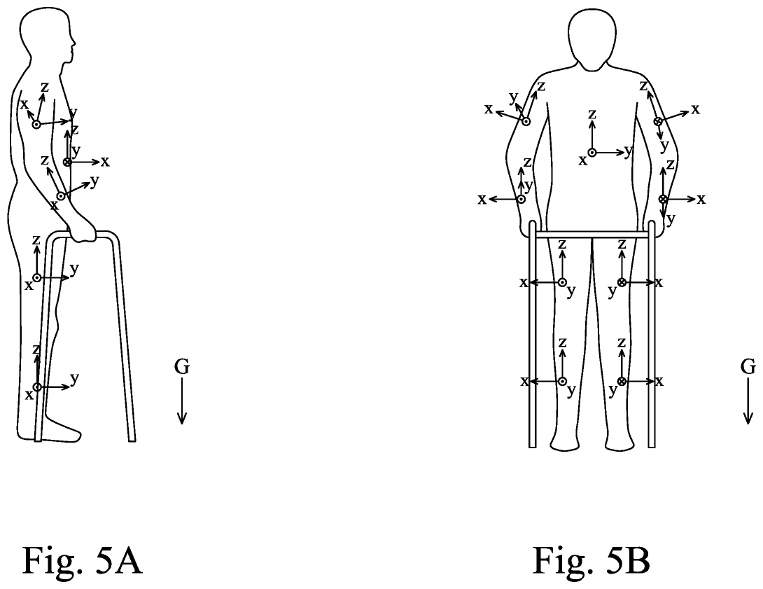

If the motion sensors detect the characteristic values of the posture configuration of standing with support, as shown in FIG. 5A and FIG. 5B, the operating unit of posture determination will select the posture in the evaluation item A-2 “Standing with support”, or the posture in the evaluation item B-11 “Standing up to sitting down”, or the posture in the evaluation item B-12 “Standing and picking up a pencil on the floor” in Table 1 for the subsequent postural control ability evaluations. These evaluation items include the following evaluation conditions:

A-2. cannot stand, even with support; can stand with strong support of two persons; can stand with moderate support of one person; can stand with support on only one hand;
B-11. cannot perform the activity; can perform the activity with much help; can perform the activity with a little help; can perform the activity without help; and
B-12. cannot perform the activity; can perform the activity with much help; can perform the activity with a little help; can perform the activity without help.

### Embodiment 3. Standing without Support

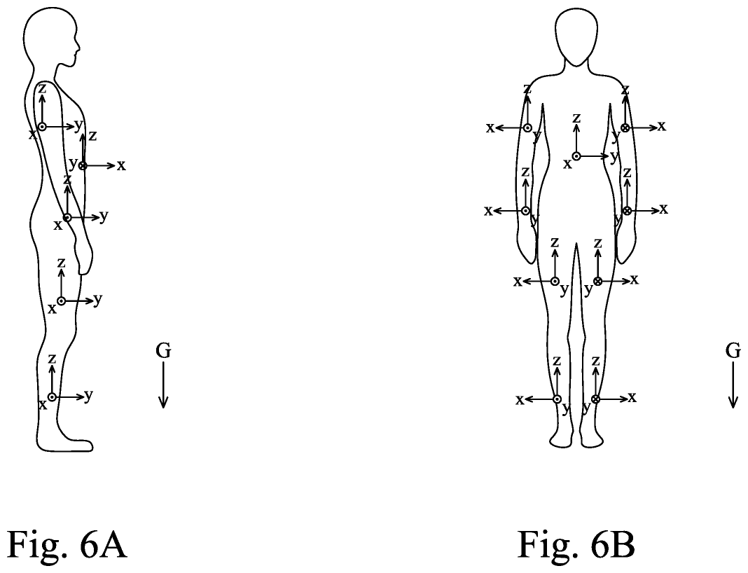

If the motion sensors detect the characteristic values of the posture configuration of standing without support, as shown in FIG. 6A and FIG. 6B, the operating unit of posture determination will select the posture in the evaluation item A-3 “Standing without support” in Table 1 for the subsequent postural control ability evaluation. The evaluation item includes the following evaluation conditions:

A-3. cannot stand without support; can stand without support for more than 10 seconds or leans heavily on one leg; can stand without support for more than 1 minute or stands slightly asymmetrically; can stand without support for more than 1 minute and perform arm movements above the shoulder at the same time.

During A-3 evaluation, the motion sensors will measure the dynamic sensing data such as the unintended tilting, jitter and swing of the trunk and each limb to analyze the subject's postural control ability under this standing posture accordingly. If the measured dynamic sensing data cannot correspond to the defined characteristic value of the standing posture configuration, it can objectively reflect the fact that the subject cannot stand without support.

### Embodiment 4. Standing on the Unaffected or Non-Paretic Leg (or Left Leg)

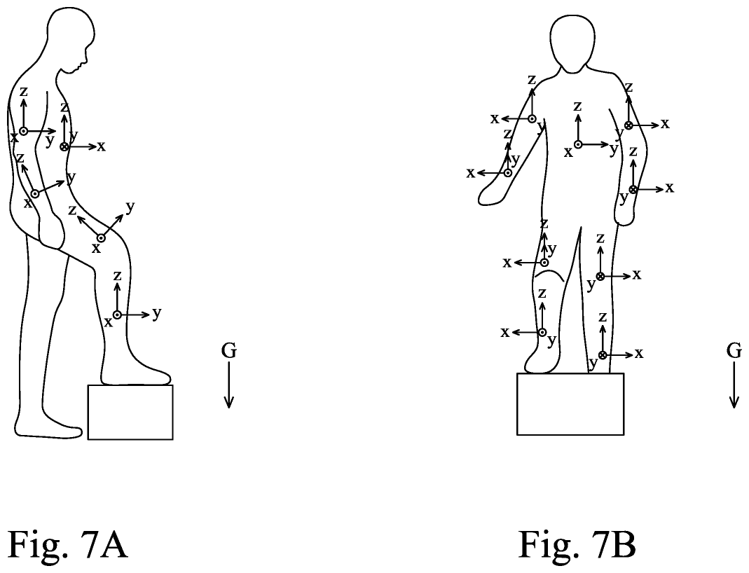

If the motion sensors detect the characteristic values of the posture configuration of standing on the unaffected or non-paretic leg (or left leg), as shown in FIG. 7A and FIG. 7B, the operating unit of posture determination will select the posture in the evaluation item A-4 “Standing on the unaffected/non-paretic leg (or left leg)” in Table 1 for the subsequent postural control ability evaluation. The evaluation item includes the following evaluation conditions: A-4. cannot stand; can stand for a few seconds; can stand for more than 5 seconds; can stand for more than 10 seconds.

During A-4 evaluation, the motion sensors will measure the dynamic sensing data such as the unintended tilting, jitter and swing of the trunk and each limb to analyze the subject's postural control ability under this body posture (i.e. under the posture of standing on the unaffected/non-paretic leg (or left leg)) accordingly. If the measured dynamic sensing data cannot correspond to the defined characteristic value of the body posture configuration, it can objectively reflect the fact that the subject cannot achieve or maintain the posture of standing on the unaffected/non-paretic leg (or left leg).

### Embodiment 5. Standing on the Affected or the Paretic Leg (or Right Leg)

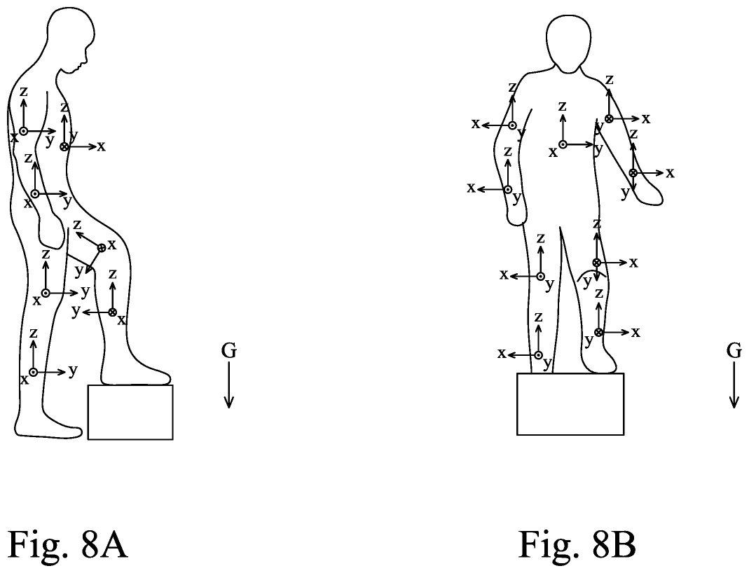

If the motion sensors detect the characteristic values of the posture configuration of standing on the affected or paretic leg (or right leg), as shown in FIG. 8A and FIG. 8B, the operating unit of posture determination will select the posture in the evaluation item A-5 “Standing on the affected/paretic leg (or right leg)” in Table 1 for the subsequent postural control ability evaluation. The evaluation item includes the following evaluation conditions:

A-5. cannot stand; can stand for a few seconds; can stand for more than 5 seconds; can stand for more than 10 seconds.

During A-5 evaluation, the motion sensors will measure dynamic sensing data such as the unintended tilting, jitter and swing of the trunk and each limb to analyze the subject's postural control ability under this body posture (i.e. under the posture of standing on the affected/paretic leg (or right leg)) accordingly. If the measured dynamic sensing data cannot correspond to the defined characteristic value of the body posture configuration, it can objectively reflect the fact that the subject cannot achieve or maintain the posture of standing on the affected/paretic leg (or right leg).

### Embodiment 6. Supine Posture

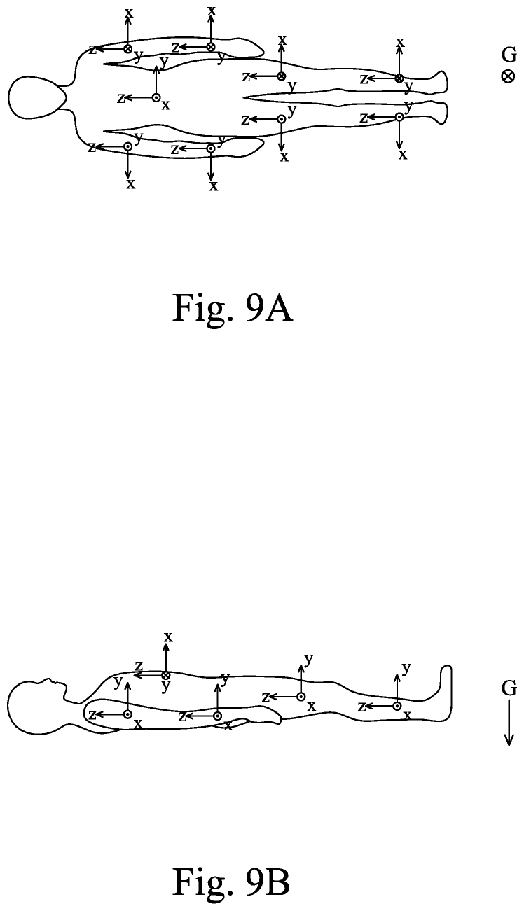

If the motion sensors detect the characteristic values such as the angles or the orientations of the trunk or the limbs of the body of in supine posture configuration, as shown in FIG. 9A and FIG. 9B, the operating unit of posture determination will select the posture in the evaluation item B-6 “Supine posture to affected/paretic side (or left side)”, or the posture in the evaluation item B-7 “Supine posture to unaffected/non-paretic side (or right side)”, or the posture in the evaluation item B-8 “Supine posture to sitting up” in Table 1 for the subsequent postural control ability evaluations. These evaluation items include the following evaluation conditions:

B-6. cannot perform the activity; can perform the activity with much help; can perform the activity with a little help; can perform the activity without help;
B-7. cannot perform the activity; can perform the activity with much help; can perform the activity with a little help; can perform the activity without help; and
B-8. cannot perform the activity; can perform the activity with much help; can perform the activity with a little help; can perform the activity without help.

The motion sensors in the present invention can sense the angles or the orientations data and the motion sensing data of the trunk and each limb under the subject's initial body posture (e.g. supine posture) and final body posture (e.g. lying on the left, lying on the right, or sitting posture), and correspond the measured data to the defined characteristic value of the supine posture configuration and the defined characteristic value of the final posture configuration, so as to obtain the postural control abilities of these body posture changes.

It is understood, that this invention is not limited to the particular embodiments disclosed, but is intended to cover all modifications which are within the spirit and scope of the invention as defined by the appended claims, the above description, and/or shown in the attached drawings.

What is claimed is:

1. A device for evaluating a postural control ability of a subject, comprising:
a sensing unit comprising a plurality of motion sensors correspondingly configured on a plurality of body parts of the subject to generate a plurality of sensing data, wherein each of the plurality of sensing data comprises static sensing data and dynamic sensing data;
a database for storing a reference value of posture maintenance of each body part under a specific one of a plurality of referencing body postures; and
a processor configured to receive the plurality of sensing data from the sensing unit and the reference value of posture maintenance from the database, and comprising:
a first operating unit of posture determination configured to form a body posture configuration according to the plurality of static sensing data, so as to determine a specific static body posture of the subject, wherein the specific static body posture corresponds to the specific one of the plurality of referencing body postures; and
a second operating unit of evaluating the postural control ability configured to process the plurality of dynamic sensing data to obtain a posture maintenance parameter for a specific one of the plurality of body parts, and compare the posture maintenance parameter with the corresponding reference value of posture maintenance of the specific body part under the specific static body posture, so as to generate an evaluation result of the postural control ability.

2. The device as claimed in claim 1, further comprising:
a display coupled to the processor for displaying the evaluation result of the postural control ability.

3. The device as claimed in claim 1, wherein the processor is wirelessly or electrically connected to an input device having an interface, and the input device is configured to receive an input of an operator and control the processor via the interface.

4. The device as claimed in claim 1, wherein the subject is one selected from a group consisting of a healthy subject, a patient, a wounded and a mobility impaired person.

5. The device as claimed in claim 1, wherein the plurality of body parts comprise trunk, upper arms, wrists, thighs and calf, and the sensing unit further comprises a support sensor for sensing a contact from a supporter.

6. The device as claimed in claim 1, wherein the static sensing data comprise an angle or an azimuth angle of the specific body part relative to a reference direction, and the dynamic sensing data comprise at least two of an acceleration, an angular velocity, a displacement and a frequency.

7. The device as claimed in claim 6, wherein the first operating unit of posture determination is configured to form a plurality of vectors according to the plurality of angles or the plurality of azimuth angles of respective ones of the plurality of body parts, and the plurality of vectors constitute a matrix model or a vector model representing the specific static body posture correspondingly.

8. The device as claimed in claim 1, wherein the database is further configured to store an evaluation content including a plurality of evaluation items and a plurality of evaluation conditions, the evaluation content is an assessment scale, the plurality of evaluation items include a plurality of sitting evaluation items and a plurality of standing evaluation items, and the plurality of evaluation conditions include a maintenance ability and an assistance degree for the plurality of evaluation items.

9. The device as claimed in claim 1, wherein the plurality of referencing body postures comprise a standing posture, a sitting posture, a squatting posture, a prone posture and a lying posture.

10. The device as claimed in claim 1, wherein the reference value of posture maintenance comprises a reference maintenance time, a reference deflection angle, a reference jitter time, a reference jitter amplitude and a reference jitter frequency.

11. The device as claimed in claim 1, wherein the posture maintenance parameter comprises a maintenance time, a deflection angle, a jitter time, a jitter amplitude and a jitter frequency.

12. A method for evaluating a postural control ability of a subject, comprising the steps of:
configuring a plurality of motion sensors on a plurality of body parts of the subject correspondingly to generate a plurality of sensing data, wherein each of the plurality of sensing data comprises static sensing data and dynamic sensing data;
performing a posture determination procedure according to the plurality of static sensing data to determine a specific body posture of the subject; and
performing an evaluation procedure for the postural control ability, wherein the evaluation procedure comprises:
processing each of the plurality of dynamic sensing data to obtain a posture maintenance parameter for each of the plurality of body parts under the specific body posture; and
comparing the posture maintenance parameter with a reference value of posture maintenance for each of the body parts under the specific body posture so as to generate an evaluation result of the postural control ability.

13. The method as claimed in claim 12, wherein the specific body posture is a specific static body posture or a dynamic body posture, and the dynamic body posture is a state that starts from the specific static body posture.

14. The method as claimed in claim 12, wherein the posture determination procedure comprises forming a body posture configuration according to the plurality of static sensing data, and comparing the body posture configuration to an evaluation content including a plurality of evaluation items and a plurality of evaluation conditions, to determine a corresponding evaluation item for the body posture configuration.

15. The method as claimed in claim 14, further comprising:
comparing the evaluation result of the postural control ability to the evaluation content;
determining a corresponding evaluation condition for the evaluation result of the postural control ability;
selecting another evaluation condition according to the evaluation result of the postural control ability; and
generating another evaluation result of the postural control ability.

16. The method as claimed in claim 12, further comprising re-performing the posture determination procedure and the evaluation procedure for the postural control ability to verify the evaluation result of the postural control ability.

17. The method as claimed in claim 12, further comprising suggesting an action practice planning according to the evaluation result of the postural control ability, and the action practice planning comprises a plurality of different action practice contents and a corresponding time allocation for each of the plurality of different action practice contents.

18. A method for evaluating a postural control ability of a subject, comprising the steps of:
configuring a plurality of motion sensors on a plurality of body parts of the subject correspondingly to generate a plurality of dynamic sensing data; and
performing an evaluation procedure for the postural control ability under a specific body posture, wherein the evaluation procedure comprises:
processing each of the plurality of dynamic sensing data to obtain a posture maintenance parameter for each of the plurality of body parts under the specific body posture; and
comparing the posture maintenance parameter with a reference value of posture maintenance for each of the body parts under the specific body posture, so as to generate an evaluation result of the postural control ability.

19. The method as claimed in claim 18, wherein the plurality of motion sensors are further configured to generate a plurality of static sensing data.

20. The method as claimed in claim 19, further comprising performing a posture determination procedure according to the plurality of static sensing data to determine a specific static body posture of the subject.
